(19) United States
(12) Patent Application Publication
(10) Pub. No.: US 2006/0225741 A1
(43) Pub. Date: Oct. 12, 2006

(54) Method and apparatus for aiding in the proper insertion and positioning of an endotracheal tube

(76) Inventor: Glenn Rothman, Scottsdale, AZ (US)

Correspondence Address: SNELL & WILMER;ONE ARIZONA CENTER, 400 EAST VAN BUREN, PHOENIX, AZ, 85004-2202, US

(21) Appl. No.: 11/385,138

(22) Filed: Mar. 20, 2006

Related U.S. Application Data
(60) Provisional application No. 60/663,478, filed on Mar. 18, 2005.

(51) Int. Cl. A62B 9/06; A61M 16/00

(52) U.S. Cl. 128/207.14; 128/200.26; 128/207.15; 128/207.16

(57) ABSTRACT

A method and apparatus for aiding in the proper insertion and positioning of an endotracheal tube includes a first long, flexible, hollow tube, a second short, hollow tube having a tapered end, and a hollow connector capable of connecting the first and second tubes.

### CROSS-REFERENCE TO RELATED APPLICATIONS

This application claims the benefit of, and priority to U.S. Provisional Application No. 60/663,478, filed Mar. 18, 2005, which application is hereby incorporated by reference in its entirety.

### FIELD OF INVENTION

The present invention generally relates to a method and apparatus for aiding in the proper insertion and positioning of an endotracheal tube and, more particularly relates to a method and apparatus for aiding in the proper insertion and positioning of an endotracheal tube which is comprised of three separate components which include a long flexible hollow tube, a short, hollow tube having a greater diameter than the long, flexible hollow tube and a tapered end, and a hollow connector which connects the long, flexible, hollow tube and the short, hollow tube having a tapered end.

### BACKGROUND OF THE INVENTION

It is critical to maintain a patient's airway and assure that proper ventilation/oxygenation is taking place. Endotracheal tubes are used to ventilate patients who may be conscious, semi-conscious or unconscious. The conventional approach to inserting an endotracheal tube involves the insertion of a rigid laryngoscope blade into a patient's mouth and pharynx. However, conventional intubation by direct laryngoscopy can be difficult and sometimes impossible. Accordingly, there is a need for an improved method and apparatus for aiding in the proper insertion and positioning of an endotracheal tube.

### SUMMARY OF THE INVENTION

The present invention is directed to an apparatus for inserting and positioning an endotracheal tube which includes a first long, flexible, hollow tube, a second short, hollow tube having a tapered end, and a hollow connector capable of connecting the first and second tubes to one another. The tapered end of the second tube is configured so that it is capable of fitting into a suction tubing, and in particular any standard suction tubing. The connector has a first end and a second end and the diameter of the first end of a connector may be smaller than the diameter of the second end of the connector.

The present invention is also directed to a method for inserting and positioning an endotracheal tube which includes the steps of providing a first long, flexible, hollow tube, a second short, hollow tube having a tapered end, and a hollow connector; connecting an end of the first tube and a non-tapered end of the second tube with the connector; connecting suction tubing to the tapered end of the second tube; inserting an open, unconnected end of the first tube into a patient's trachea; removing the second tube from the connector; attaching an open, unconnected end of the connector to a ventilator to ensure proper positioning of the first tube within the patient's trachea; removing ventilation from the connector and removing the connector from the first tube; sliding an endotracheal tube having a diameter larger than a diameter of the first tube over the first tube and into the patient's trachea; and removing the first tube from the patient's trachea by pulling it out through the endotracheal tube.

### BRIEF DESCRIPTION OF THE DRAWINGS

The subject invention will hereinafter be described in conjunction with the appended drawing figures, wherein like numerals denote like elements, and

FIG. 1 is a perspective view of the three separate components of the invention shown disconnected;

FIG. 2 is a perspective view of the first and second components of the invention shown connected;

FIG. 3 is a perspective view of all three separate components of the invention shown connected.

### DETAILED DESCRIPTION

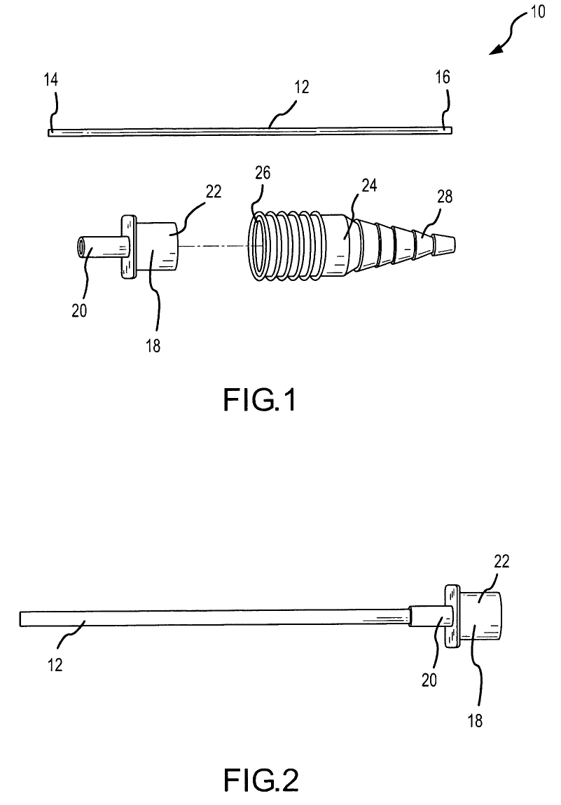

The present invention is directed to a method and apparatus for aiding in the proper insertion and positioning of an endotracheal tube. As shown in FIG. 1, the apparatus 10 comprises three components. The first component 12 is a long, flexible, hollow tube having first and second ends 14, 16. The second component 18 is a hollow connector which comprises a first end 20 and a second end 22 where the diameter of the first end 20 is smaller than the diameter of the second end 22 and the diameter of the first end 20 fits over an end 14, 16 of the long, flexible hollow tube that comprises the first component 12. The third component 24 comprises a short, hollow tube having a first end 26 with a diameter that fits over the second end 22 of the connector that comprises the second component 18 and a second tapered end 28 that comprises a cone shape capable of fitting into any type of standard sized suction tubing.

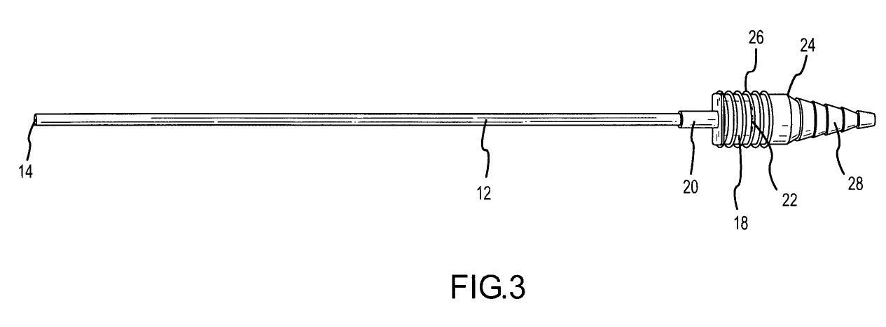

In order to use the device to properly insert and position an endotracheal tube, the first end 20 of the connector (second component) 18 is fitted over a first or second end 14, 16 of the first component 12 (See FIG. 2). The first end 26 of the third component 24 is then fitted over the second end 22 of the connector (second component) 18 (See FIG. 3). Suction tubing (not shown) is then fitted over the second tapered end 28 of the third component 24 and the open and unconnected end of the first component 12 is then inserted into a patient's trachea. Once inserted, the third component 24 is removed from the connector (second component) 18 and the second end 22 of the connector is attached to a ventilator to ensure proper positioning of the first component within the patient's trachea. If positioned properly, ventilation is removed and the connector is removed from first component (i.e. the long tube) and an endotracheal tube having a diameter larger than the first component 12 is slid over the first component 12 and into the patient's trachea. The first component 12 is then removed from the patient by pulling it out of the person's trachea through the endotracheal tube.

In one exemplary embodiment, the first component may comprise a flexible, hollow tube made of plastic where the tube is approximately 24 inches long with about a 6 mm diameter and the second component may comprise a connector where the first end of the connector is equivalent to the connector to a 5.5 Fr endotracheal tube circuit connector.

The device may also be used to achieve the same end result (i.e. an endotracheal tube in the trachea) by passing the first end through a laryngeal mask airway (LMA) tube already in the pharynx. The connectors are used to establish temporary ventilation and confirm that the tube is indeed in the trachea. The two connectors are then removed and the LMA is withdrawn over the long tube which remains in the trachea. An endotracheal tube is then passed over the long tube as described in the first scenario.

Although the invention has been described herein in conjunction with the appended drawings, those skilled in the art will appreciate that the scope of the invention is not so limited. Modifications in the selection, design, and arrangement of the various components and steps discussed herein may be made without departing from the scope of the invention.

1. An apparatus for inserting and positioning an endotracheal tube comprising:
A first long, flexible, hollow tube;
A second short, hollow tube having a tapered end; and
A hollow connector capable of connecting the first and second tubes.

2. The apparatus of claim 1 wherein the tapered end of the second tube is capable of fitting into a suction tubing.

3. The apparatus of claim 1 wherein the connector has a first end and a second end and a diameter of the first end is smaller than a diameter of the second end.

4. The apparatus of claim 3 wherein the first end of the connector is connected to the first tube and the second end of the connector is connected to the second tube.

5. The apparatus of claim 4 wherein the tapered end of the second tube is capable of fitting into a suction tubing.

6. A method for inserting and positioning an endotracheal tube comprising the steps of:
a) providing a first long, flexible, hollow tube, a second short, hollow tube having a tapered end, and a hollow connector;
b) connecting an end of the first tube and a non tapered end of the second tube with the connector;
c) connecting suction tubing to the tapered end of the second tube;
d) inserting an open, unconnected end of the first tube into a patient's trachea;
e) removing the second tube from the connector;
f) attaching an open, unconnected end of the connector to a ventilator to ensure proper positioning of the first tube within the patient's trachea;
g) removing ventilation from the connector and removing the connector from the first tube;
h) sliding an endotracheal tube having a diameter larger than a diameter of the first tube over the first tube and into the patient's trachea; and
i) removing the first tube from the patient's trachea by pulling it out through the endotracheal tube.

7. The method of claim 6 wherein the step of providing a first long, flexible, hollow tube, a second short, hollow tube having a tapered end, and a hollow connector comprises the step of providing a connector having a first end and a second where a diameter of the first end is smaller than a diameter of the second end.

8. The method of claim 7 wherein the step of connecting an end of the first tube and a non tapered end of the second tube with the connector comprises the step of connecting the first end of the connector to the first tube and the second end of the connector to the second tube.
